(19) United States
(12) Patent Application Publication
(10) Pub. No.: US 2003/0153053 A1
(43) Pub. Date: Aug. 14, 2003

(54) Methods for altering polyketide synthase genes

(76) Inventor: Ralph Reid, San Rafael, CA (US)

Correspondence Address: Kosan Biosciences, Inc., 3832 Bay Center Place, Hayward, CA, 94545, US

(21) Appl. No.: 10/214,424

(22) Filed: Aug. 6, 2002

Related U.S. Application Data
(60) Provisional application No. 60/310,778, filed on Aug. 6, 2001.

(51) Int. Cl. C12P 19/62; C12N 9/10; C12P 21/02; C12N 1/21; C12N 15/74

(52) U.S. Cl. 435/76; 435/252.3; 435/471; 435/69.1; 435/320.1; 435/193

(57) ABSTRACT

Ketoreductase (KR) domains of modular polyketide synthase (PKS) enzymes can be inactivated by one or more point mutations in the domain. Replacement or insertion of a KR domain can be used to introduce a cis or trans double bond into a polyketide by appropriate selection or inactivation of the type of KR domain that codes for a particular stereochemical configuration of a hydroxyl moiety. Inactivation of a DH domain can be used to produce a polyketide having a hydroxyl moiety with a desired stereochemical configuration.

### CROSS-REFERENCE TO RELATED APPLICATIONS

This application claims the benefit of the filing date under 35 USC §119(e) with respect to U.S. Provisional application 60/310,778, filed Aug. 6, 2001. The disclosure of this application is incorporated herein by reference.

### TECHNICAL FIELD

This invention relates to methods for manipulating specific modules of a modular polyketide synthase such that the resulting polyketide has an altered stereochemistry or chemical structure.

### BACKGROUND OF THE INVENTION

The present application provides methods to predict the stereospecificity of the ketoreductases (KRs) of modular polyketide synthases (PKSs) from protein sequence; methods to alter PKS genes to inactivate or change the stereospecificity of a KR; methods to provide a PKS with a KR of a desired stereochemical specificity; methods to predict the dehydration specificity (cis vs. trans) of polyketide modules with active dehydratase (DH) domains; and methods to produce a cis double bond by PKS gene alteration.

Modular PKSs control the structure and stereostructure of their products using families of related domains: these domains have evolved to have varied substrate specificity and varied stereochemical pathways. Ketoreductase domains have been shown to control the stereospecificity of the two observed alcohol stereochemical possibilities; enoyl reductase domains are expected to control certain cases of side chain stereoposition; the control of other side chain stereopositions is currently unclear; and a degree of control of cis vs. trans stereochemistry is believed by some to reside in dehydratase domains. Sequence analysis of domain families, compared with available structures of related proteins, can be used to predict the structural basis of this variety. Proposals on the mechanism of ketoreductase domains can be used to predict polyketide stereochemistry from ketoreductase sequences; these predictions have implications for the mechanism of dehydratases. The present invention relates to methods for altering the product of a PKS by KR domain alteration.

To aid in comprehending the present invention, relative stereochemistry terminology is provided that is useful to describe similarities and differences in biochemical pathways involving linear polymers such as polyketides and in the stereochemistry along a designated directed subchain (the “backbone”) in a network of organic chemical type in 3D-space (with a primary interest being in designating which of two tetrahedral configurations is present at relevant nodes). In particular, this terminology facilitates discussing polyketides (PKs) and the biosynthesis of polyketides by PKSs. Many PKS proteins are large and multifunctional, composed of domains with separate enzymatic activities. The stereochemical course of such a domain's reaction mechanism may not be well described by standard Cahn-Ingold-Prelog terminology applied to the substrate, as the priority of various atoms in that teminology may be determined by variable substituents distant from (or at least not relevant to) the site of interest. For example, at the indicated (“site”) carbonyl carbon in the structure below, reduction by the ketoreductase (KR) activity of a particular PKS domain may occur from above the page, producing a hydroxyl with stereochemistry of “type 1” (as defined below), as in the product of module 1 of DEBS; or from below, producing “type 2”, as produced by extender modules 2, 5 and 6 of DEBS.

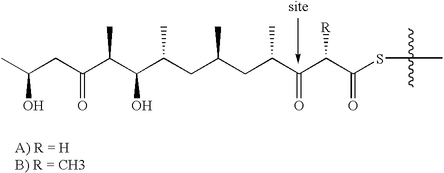

In the above example, the face of attack and the product are described differently by Cahn-Ingold-Prelog terminology depending on the substituent R. If R═H, attack from “above” is at the re face, and produces the S configuration. If R═CH3, then it is at the si face and produces the R configuration. The same KR engineered in two different polyketide modules is expected to attack from a constant direction relative to the growing polyketide backbone. The terminology defined below gives a standard way of describing such directions.

The relative stereochemistry terminology used herein provides that, given a graph G, composed of vertices (“atoms”) and edges (“bonds”) in a particular conformation in 3D-space, one can assume the graph is of “bioorganic” type (no more than 4 bonds/vertex; double and triple bonds allowed). Let B (for “backbone”) be a directed non-self-intersecting connected path within G. The path (“backbone”) is assumed to resemble a chain restricted to carbons and nitrogens in the following manner: if there are 4 bonds from a vertex on the path to separate vertices, then the conformation is assumed to be non-planar at that site.

Priorities at tetrahedral vertices of B are assigned as follows: priority 1, the forward direction; priority 2, the reverse direction; priorities 3 & 4, as in Cahn-Ingold-Prelog. The two possibilities are designated R_{B} and 5_{B} (“R relative to the backbone B” and “S relative to the backbone B”).

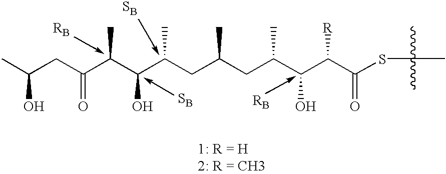

At a general tetrahedral backbone position, an external substituent direction is designated pro-R_{B} (or “type 1”), if priority in that direction would give RB as the configuration; and pro-5_{B} (or “type 2”), if priority in that direction would give SB as the configuration. Thus a gem-dimethyl site will have one methyl of type 1 and one of type 2.

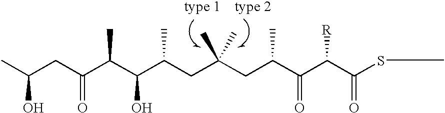

At trihedral vertices with a single external bonded atom coplanar with the bonds of the path (e.g., carbonyl sites or at double bonds in the path), the priorities are assigned as: priority 1, the direction of the external bonded atom; priority 2, the forward direction; priority 3, the reverse direction. The faces are designated re_{B} and si_{B} (“relative” re and “relative” si). Thus in the initial example, the upper face is always si_{B} and the lower face always re_{B}.

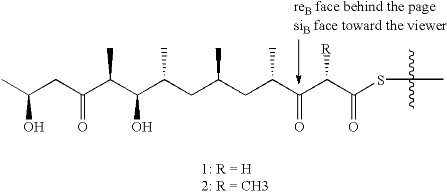

At double bonds within the path, the higher priorities go to the bonds along the path. The resulting possibilities are designated E_{B} and Z_{B} (“relative” trans and “relative” cis).

### SUMMARY OF THE INVENTION

In one embodiment, the present invention provides a method to inactivate a ketoreductase (KR) domain in a modular polyketide synthase (PKS), said method comprising changing one or more conserved amino acids in a short chain dehydrogenase/reductase (SDR) active site motif to another amino acid, wherein said SDR active site motif is defined by an amino acid sequence: HX˜₆DX˜₁₆₋₁₈KX_{˜26}SSX˜₁₂YX˜₃N, wherein X is any amino acid followed by a subscript indicating a number of amino acids between two conserved residues, and wherein said conserved amino acid that is changed is selected from the group consisting of K, S, S, and Y. The desired changes can be effectuated by altering a coding sequence in a gene encoding said KR.

It should be noted that one skilled in the art can recognize, target, and manipulate, using techniques known to one skilled in the art, motifs such as the one described above.

In another embodiment, the invention provides a method to alter a module of a modular PKS such that said module will introduce a cis double bond into a polyketide produced by said PKS, said method comprising, either (A) replacing an entire module for the position at which the cis double bond is desired with a module having a type 2 KR and dehydratase (DH) domains, (B) exchanging a portion of a module between an AT and an ACP of said module for a DH plus a type 2 KR domain of another module, (C) in a module already producing a trans double bond, replacing a type 1 KR domain with a type 2 KR domain, (D) in a module containing a type 1 KR domain, changing the KR to a type 2 KR domain by point mutation or replacing the KR with a type 2 KR; and (E) inserting a DH into a module containing a type 2 KR.

In another embodiment, the present invention provides a method for introducing a hydroxyl moiety having a particular stereochemical configuration in a polyketide by inactivating a DH domain adjacent to a type 1 or type 2 KR domain.

These and other embodiments of the invention are described more fully in the Brief Description of the Figures, Detailed Description of the Invention, and claims that follow.

### BRIEF DESCRIPTION OF THE DRAWINGS

FIG. 1A shows the traditional SDR catalytic triad.

FIG. 1B shows a sequence motif common to standard SDR active site residues.

FIG. 1C shows a sequence motif common to ketoreductases from processive modular PKSs.

FIG. 2 shows the cofactor, product, and active site residues from a TRII ternary product complex.

### DETAILED DESCRIPTION OF THE INVENTION

The present invention arose in part from an appreciation of how an analysis based on the crystal structures, together with site-directed mutagenesis, of two highly homologous (64% identity) tropinone ketoreductases, might have application to polyketide biosynthesis. This analysis, based on work with the tropinone ketoreductases, appeared in PNAS 95: 4876-81 (1998) and JBC 274: 16563-8 (1999), each by Nakajima and collaborators, together with Biochemistry 38: 7630-7637 (1999) by Yamashita et al., each of which is incorporated herein by reference. The two tropinone ketoreductase enzymes share a common substrate and common stereospecificity with respect to the cofactor NADPH (transferring the pro-S-hydrogen from a nicotinamide in syn conformation), but the alcohol products have opposite configurations (S vs. R).

Studies of chimeras, mutants and homologs focused attention on 5 residues. These were mutated singly and in various combinations by Najima et al., changing each residue to the equivalent one in the other enzyme. The change of all 5 completely changes stereospecificity; this allows change of either of the enzymes to the specificity of the other. Two substrate configurations are expected; in one of them, a side chain at one of the five sites in particular (His112 vs. Tyr100) is predicted to interact with a heteroatom (N) of the substrate that is separated by two carbons from the carbon of the carbonyl reduced. Crystal structures can be used to predict a mechanism in which a conserved tyrosine and a serine both hydrogen-bond to the oxygen of the carbonyl, one of them providing a proton during reduction. In addition, a lysine a few residues to the C-terminal side of the tyrosine is involved in binding of the sugar hydroxyls of the nicotinamide portion of the cofactor.

The tropinone reductases fall into a large family of nicotinamide cofactor-dependent reductases known as the SDR superfamily, which includes the reductases of eubacterial type II fatty acid synthases, including that of E. coli. Over 1000 members have been assigned to this family by Jornvall et al., FEBS Letters 445: 261-264 (1999), and references therein, incorporated herein by reference. The family has a Rossmann fold at the N-terminus, which the crystal structure confirms is the binding site of the adenosine-pyrophospho portion of the cofactor. In this family, Ser-Tyr-Lys active site residues are highly conserved. The function of the charge of the Lys is speculative, but this residue is believed to contribute to the acidity of the transferred hydrogen.

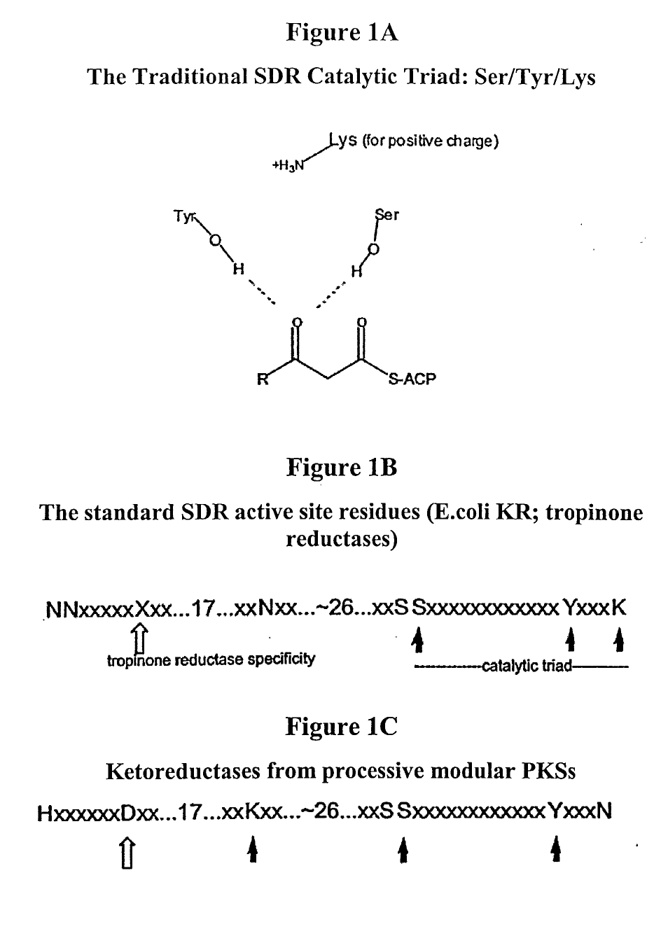

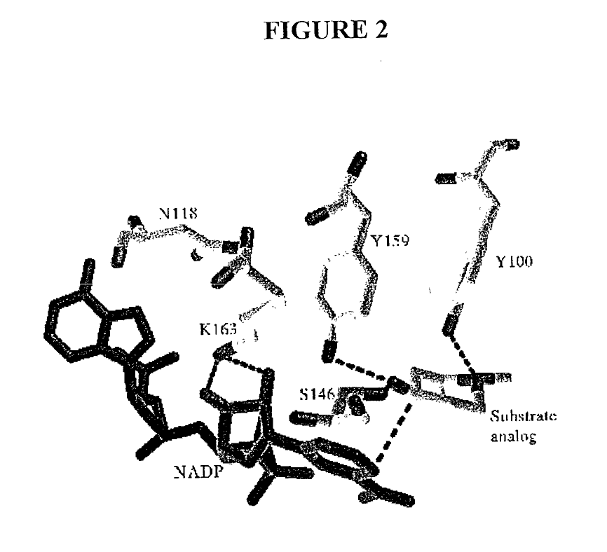

In accordance with the methods of the present invention, the family of protein sequences of ketoreductases of modular polyketides can be aligned with the sequences of this superfamily, and in particular with those of the tropinone reductases. At the N-termini, a Rossmann fold region corresponds to the SDR Rossmann fold. An absolutely conserved Tyr corresponds to the SDR conserved Tyr; an absolutely conserved Asn corresponds to the Lys. An absolutely conserved Lys in the ketoreductase family corresponds to a very highly conserved Asn in the SDR superfamily generally, including the tropinone reductases; this Asn in the the tropinone reductase crystal structures is very near the tropinone reductase conserved Lys. Corresponding to the Ser site, there is often a pair of adjacent serines, and one of the two is always present in 168 of 169 analyzed KR domains. The ketoreductases of human and other Animalian (vertebrate and invertebrate) type I fatty acid synthases correspond to this modular polyketide type (in particular, with the conserved Lys and Asn reversed from the general SDR pattern), as shown in FIG. 1.

FIG. 1A shows the traditional SDR catalytic triad: Ser/Tyr/Lys. FIG. 1B shows a sequence motif, common to standard SDR active site residues, taken from E. coli KR, and tropinone reductases, among others. Arrows represent regions specific for tropinone reductase specificity and a catalytic triad. FIG. 1C shows a sequence motif common to over 200 ketoreductases from processive modular PKSs.

Figure two shows a molecular model of the cofactor, product, and active site residues from a TRII ternary product complex. Shown are specific amino acids and their locations and sites for NADP and a substrate analog.

The following is a list of commonly known symbols of amino acids:

| | | |
|---|---|---|
| A | Ala | Alanine |
| B | Asx | Asparagine or aspartic acid |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Trptophan |
| X | | any amino acid |
| Y | Tyr | Tyrosine |
| Z | Glx | Glutamine or glutamic acid |

The present invention arose in part from an appreciation that the proton transferred during ketoreduction by modular polyketide synthases comes from a network involving direct interaction with both the OH of the conserved Tyr and an OH from a Ser at one of the two adjacent Ser-rich sites. Thus, in accordance with the methods of the present invention, one can, for example, replace the Tyr with Phe or another amino acid and the appropriate Ser(s) with Ala(s) or other amino acid(s) to inactivate a KR by point mutation (while minimally disrupting its structure). In accordance with the present invention, it is believed that the conserved Lys of the modular polyketide KRs (replacing a nearby SDR conserved Asn) provides the positive charge provided by the conserved SDR Lys. Therefore, in another embodiment, the invention provides a method to inactivate a ketoreductase by point mutation by replacing this Lys by another amino acid, either singly or in combination with alterations discussed above. This aspect of the invention is illustrated in Example 1, below.

The present invention also provides methods for altering the sterochemistry and type of double bond (cis or trans) formed in polyketides by manipulation of KR domains. In the family of ketoreductases of modular polyketides, as in the tropinone ketoreductase family, two alternate modes of binding are expected. In the homologous Animalian FAS ketoreductases, the stereochemistry of reduction is known to be that which would produce the conformation of the hydroxy of module 1 of DEBS; see for example Biochemistry 23: 2088-2094 (1984) Anderson and Hammes, incorporated herein by reference. In many polyketide modules, including modules 2, 5 and 6 of DEBS, the opposite stereoisomeric conformation is produced. For still others, in which the modules have active DH and sometimes ER domains, the KR stereospecificity has been generally unknown.

When the various KRs from PKSs are aligned in accordance with the methods of the present invention, they can be grouped into two different families. At the site corresponding to the His112 vs. Tyr100 of the tropinone reductases, Animalian FAS sequences have a conserved Asp. In all modular PKS KRs of type 1, this Asp is also conserved. In all modular PKS KRs of type 2, it is absent.

In accordance with the methods of the present invention, this site is diagnostic of the stereochemistry of such KRs. The substrates of the modular PKS KRs are acyl-ACPs, with two heteroatoms (S and a carbonyl oxygen) each separated by two carbons from the carbonyl of the reduction, analogously to the tropinone configuration. In accordance with the invention, it is believed that the Asp interacts with one or both of these in the substrate conformations required for type 1 reduction; and that type 2 reduction would tend to be interfered with the presence of such a residue (which would tend in that case to stabilize inappropriate conformations of interaction).

When the “silent” KRs from modules that produce double bonds are aligned, almost all published sequences fall into type 1 (as in the Animalian FAS cases). The only exceptions are also the only cases in which the DH appears to produce a cis double bond at the C2-C3 position (module 10 of rifamycin and three others). See Table 1, below.

TABLE 1
CORRELATION OF STEREOCHEMISTRY WITH SEQUENCE
MOTIFS IN 169 KR DOMAINS FROM PKS POLYPROTEINS.

| | # | H | D | K | SS/SX/XS | Y | N |
|---|---|---|---|---|---|---|---|
| TYPE1 KR | 36 | 100% | 100% | 100% | 100% | 100% | 100% |
| KR −> DH | 63 | 100% | 100% | 100% | 98% | 100% | 100% |
| (trans) | | | | | | | |
| KR −> | 26 | 100% | 100% | 100% | 100% | 100% | 100% |
| DH −> ER | | | | | | | |
| TYPE2 KR | 38 | 100% | 0% | 100% | 100% | 100% | 100% |
| KR −> DH (cis) | 4 | 100% | 0% | 100% | 100% | 100% | 100% |
| other predicted | 2 | 100% | 0% | 100% | 100% | 100% | 100% |
| type2 | | | | | | | |
| TOTALS | 169 | 100% | 74% | 100% | 99.4% | 100% | 100% |

Other such known cis double bonds in polyketides with sequenced synthases are either produced by post-PKS proteins, or by other mechanisms outside the module (avermectin aveC; the epothilone mechanism at the module 4 product). The DHs from a large number of processive eubacterial polyketide synthases appear structurally homologous to each other, and to the DH domains from vertebrate fatty acid synthases, in modules producing both cis and trans double bonds. It is therefore likely that the OH leaves in the same direction with respect to the DH in both cis and trans cases. Therefore, it is believed, in accordance with the methods of the invention, that the cis double bond is formed by a combination of type 2 KR stereochemistry, followed by a DH capable of accepting a substrate with the C3-C4 bond of the backbone in a rotated conformation compared to that seen in the more common type 1 case.

This analysis serves as the basis for the methods of the invention to the production of desired cis double bonds:

A) replace an entire module for a module with the desired KR and DH domains, such as Mod10 of the rifamycin PKS;

B) exchange the portion of a module between the AT and the ACP for the DH plus KR region of a module with the desired KR and DH domains, such as Mod10 of rifamycin PKS;

C) in a module already producing a trans double bond, one might either exchange the KR domain for a KR domain known or predicted to be of type two;

D) in a module containing normally a KR predicted to be of type 1, change the specificity of the original KR by point mutation in the gene, in particular by changing the conserved Asp seen in the type 1 KR family into various alternative amino acids in that location in the sequences of known KRs of type 2, or replace that KR with a type 2 KR; and

E) insert a DH into a module containing a KR of type 2.

Conversely, these methods can be employed in reverse to eliminate a cis double bond.

As with all such cases of polyketide engineering, productive success would of course depend on the ability of any following domains (e.g., a following KS) to accept the new product.

Thus, in accordance with the methods of the invention, one can:

F) replace the portion of a module after the AT for the equivalent region of a module, such as Mod10 of rifamycin PKS; and replace the following KS with a KS from a module normally following a module producing a cis double bond, preferably but not exclusively of the type desired with respect to AT selectivity.

The success of methods C and D also depends on the ability of the original DH to function with the altered substrate; and method E requires the choice of a DH capable of dehydrating a type 2 substrate, and compatibilty with the particular substrate, in particular with the side chain provided by the AT.

The present invention is further described by the following examples. These examples are provided solely to illustrate the invention by reference to specific embodiments. These exemplifications, while illustrating certain specific aspects of the invention, do not portray the limitations or circumscribe the scope of the disclosed invention.

As will be further described below, the metes and bounds of the invention can be described on both the protein level and the encoding nucleotide sequence level.

### EXAMPLES

The following examples describe the inactivation of a modular PKS KR domain by site-directed mutagenesis.

### Example 1

### Creation of Point Mutations Using Altered Sites Mutagenesis

Three point mutations, K2426Q, S2686A, and Y2699F, were introduced individually in the KR domain of DEBS module 6. The amino acid numbers refer to the position in DEBS3 starting from the initial methionine residue. The amino acids substituted correspond to those in the identified ‘SDR active site’ motif. These point mutations were introduced in a subcloned DNA fragment using the Altered Sites mutagenesis kit (Promaga) according to the instructions.

### Example 2

### Creation of Plasmids Containing the Three Point Mutations

The subcloned fragments harboring the three different mutations were then used to introduce the mutations into the Streptomyces expression plasmid pKOS011-77 (see U.S. Pat. Nos. 6,399,789 and 6,033,883, each of which is incorporated herein by reference) and by conventional cloning procedures with restriction sites to generate expression plasmids with the mutations in the full DEBS (6-deoxyerythronolide B synthase). Plasmid pKOS198-15 contains the K2426Q substitution in DEBS3, pKOS198-16 contains the S2686A substitution in DEBS3, and pKOS198-17 contains the Y2699F substitution in DEBS3.

### Example 3

### Protoplast Transformation of Streptomyces lividans

The plasmids were then used for protoplast transformation of Streptomyces lividans K4-114 (see U.S. Pat. No. 6,177,262, incorporated herein by reference) together with pSuperBoost which enhances expression of the DEBS genes (see U.S. patent application Ser. No. 10/126,196, filed Apr. 19, 2002, incorporated herein by reference).

### Example 4

### Selection of Transformants

Transformants were selected on R5 agar plates using thiostrepton and apramycin to select for the expression plasmid and pSuperBoost, respectively. Four independent colonies from each transformation were selected to screen for polyketide production by fermentation and LC/MS. A single representative polyketide-producing colony from each transformation was then grown in 50 mL of FKA medium supplemented with 50 mg/L thiostrepton, 200 mg/L apramycin, and 10 mM sodium propionate.

### Example 5

### Determination of Polyketide Profiles and Titers

After 7 days growth at 30 degrees C., the culture supernatants were analyzed by LC/MS/MS to determine the polyketide production profiles and titers. Complex mixtures of polyketides were present in each strain and the major metabolites were identified as 6-deoxyerythronolide B (6-dEB) and 3-deoxy-3-oxo-6-deoxyerythronolide B (3-oxo-6-dEB). Authentic purified standards were used to conform the identities of these compounds. The titers of these two metabolites from each strain are as follows:

| 6-dEB | 3-oxo-6-dEB | | | |
|---|---|---|---|---|
| K2426Q | 26 | mg/L | 17 | mg/L |
| S2686A | 21 | mg/L | 9 | |
| Y2699F | <1 | mg/L | 41 | mg/L |

The production of 3-oxo-6-dEB in the mutant strains results from bypassing the KR domain of module 6. Because pKOS011-77 does not produce any detectable 3-oxo-6-dEB and produces only macrolactones with a hydroxyl group at carbon 3 (i.e. 6-dEB and 8,8a-deoxyoleandolide), these data clearly indicate that mutation of these amino acid residues significantly reduces the activity of the KR domain in module 6. In particular, the Y2699F mutation nearly completely abolishes the KR6 activity.

The invention having now been described by way of written description and examples, those of skill in the art will recognize that the invention can be practiced in a variety of embodiments and that the foregoing description and examples are for purposes of illustration and not limitation of the following claims.

Numerous modifications may be made to the foregoing systems without departing from the basic teachings thereof. Although the present invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. All publications or patent documents cited in this specification are incorporated herein by reference as if each such publication or document was specifically and individually indicated to be incorporated herein by reference.

Citation of the above publications or documents is not intended as an admission that any of the foregoing is pertinent prior art, nor does it constitute any admission as to the contents or date of these publications or documents.

What is claimed is:

1. A method to inactivate a ketoreductase domain in a modular polyketide synthase, said method comprising changing a conserved amino acid in an SDR active site motif to another amino acid, wherein said SDR active site motif is defined by an amino acid sequence: HX˜₆DX˜₁₆₋₁₈KX₋₂₆SSX˜₁₂YX˜₃N, wherein X is any amino acid followed by a subscript indicating a number of amino acids between two conserved residues, and wherein said conserved amino acid that is changed is selected from the group consisting of K, S, S, and Y.

2. The method of claim 1 wherein Y is changed to F or X.

3. The method of claim 1 wherein S is changed to A or X.

4. The method of claim 1, wherein said change is made by altering a coding sequence in a gene encoding said KR.

5. The method of claim 1 wherein K is changed to X.

6. The method of claim 5 wherein Y is changed to F or X.

7. The method of claim 6 wherein one or both S is changed to A or X.

8. The method of claim 1, wherein more than one of said conserved amino acids is changed.

9. A method to alter a module of a modular polyketide synthase (PKS) such that said module will introduce a cis double bond into a polyketide produced by said PKS, said method comprising, either (A) replacing an entire module for a module with a type 2 KR and DH domains, (B) exchanging a portion of a module between an AT and an ACP of said module for a DH plus a type 2 KR domain of another module, (C) in a module already producing a trans double bond, replacing a KR domain for a type 2 KR domain, (D) in a module containing a type 1 KR domain, changing the KR to a type 2 KR domain by point mutation or replacing the KR with a type 2 KR; and (E) inserting a DH into a module containing a type 2 KR.

10. The method of claim 9 wherein said module will introduce a cis double bond by replacing an entire module for a module with a type 2 KR and DH domains.

11. The method of claim 9 wherein said module will introduce a cis double bond by exchanging a portion of a module between an AT and an ACP of said module for a DH plus a type 2 KR domain of another module.

12. The method of claim 9 wherein said module will introduce a cis double bond by, in a module already producing a trans double bond, replacing a KR domain for a type 2 KR domain.

13. The method of claim 9 wherein said module will introduce a cis double bond by in a module containing a type 1 KR domain, changing the KR to a type 2 KR domain by point mutation or replacing the KR with a type 2 KR.

14. The method of claim 9 wherein said module will introduce a cis double bond by inserting a DH into a module containing a type 2 KR.
